(19) United States
(12) Patent Application Publication
(10) Pub. No.: US 2012/0277433 A1
(43) Pub. Date: Nov. 1, 2012

(54) PROCESS FOR THE PREPARATION OF SUBSTITUTED QUINOXALINES

(76) Inventors: Mahendranath Rao Narasimha Rao, Bangalore (IN); Attimogae Shivamurthy Harisha, Bangalore (IN); Shridhara Kanakamajalu, Bangalore (IN); Kothapalli Sundarraja Rao, Bangalore (IN); Kuppuswamy Nagarajan, Bangalore (IN)

(21) Appl. No.: 13/519,600

(22) PCT Filed: Dec. 28, 2010
(86) PCT No.: PCT/IN2010/000857

§ 371 Date: Jun. 28, 2012

(30) Foreign Application Priority Data
Dec. 29, 2009 (IN) 3005/MUM/2009

(51) Int. Cl. C07D 471/04

(52) U.S. Cl. 544/343

(57) ABSTRACT

The present invention is directed to an improved process for the preparation of substituted quinoxalines by cyclization of the corresponding dianiline in the presence of ion exchange resin.

### FIELD OF THE INVENTION

The present invention is directed to a process for the preparation of substituted quinoxalines.

### BACKGROUND OF THE INVENTION

Substituted quinoxalines are useful intermediates in the preparation of aryl fused azapolycyclic compounds-viz-Varenicline.

Varenicline tartrate salt known chemically as 7,8,9,10-tetrahydro-6,10-methano-6 H-pyrazino[2;3-h][3]benzazepine; (2 R ,3 R)-2,3-dihydroxybutanedioate (1:1), is described according to the following formula

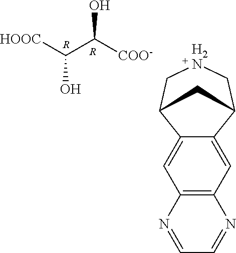

Varenicline and its pharmaceutically salts such as varenicline tartrate is marketed by Pfizer under the trade name of CHANTIX™ as a partial agonist selective for certain subtypes of nicotinic receptors and indicated for smoking cessation.

These are also are useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mat absence epilepsy, senile dementia of the Alzheimer's type (AD), Parkinson's disease (PD), attention deficit hyperactivity disorder (ADHD) and Tourette's Syndrome, particularly, nicotine dependency, addition and withdrawal; including use in smoking cessation therapy.

U.S. Pat. No 6,410,550 discloses the synthesis composition and methods of use of certain aryl fused azapolycyclic compounds including the formation of substituted quinoxaline through the cyclization of the corresponding dianiline with aqueous glyoxal or the addition adduct of sodium bisulfite and ethane dione. Both of these reactions required certain purification steps.

U.S. Pat. No. 6,890,927 and U.S. Pat. No. 7,265,119. disclose Varenicline tartrate salts (including L-tartrate, D, L-tartrate and D-tartrate salts) and their polymorphic forms. In particular, three polymorphic forms of varenicline L-tartrate, Form A, Form B and Form C were disclosed in these patents. Among them, Form A and Form B are anhydrous varenicline L-tartrate, and Form C is varenicline L-tartrate hydrate.

Crystalline. forms of Varenicline citrate and succinate salts are described in the U.S. Pat. Nos. 6,787,549 and 6,794,388, respectively.

WO 02/092089 discloses preparation of the polymorphs of the L-tartrate salt of the aryl fused azapolycyclic compound.

US2008027505 1 discloses an improved process for the preparation of substituted quinoxaline by cyclization of the corresponding dianiline with aqueous glyoxal in a protic solvent.

Thus there remains an urgent and imperative need to provide not only a more cost effective process but also a time-efficient process that would afford a superior quality product suitable for human consumption.

Surprisingly the inventors of the present invention have found that an ion exchange resin catalyses the said cyclisation reaction in an increasingly faster reaction rate with negligible side reactions and thus affords a product of superior quality in an eco-friendly and cost-effective manner.

The following are the advantages of the improved process of the present invention over the prior art:
Use of an Ion exchange resin for catalyzing Cyclisation instead of the conventional base as employed in the prior art resulted in the reduction of time Duration of the process cycle by more than 50%.
The cyclisation reaction is completed in 4-5 hr as against 18 hrs over night as reported in the prior art i.e., more than 73% of the duration is saved.
Ion exchange Resins used for catalyzing the Cyclisation reaction step can be reused and recycled by regeneration, thereby increasing the cost-effectiveness and industrial applicability of the present invention.
Resins of the like of Amberlite IRA 67 being weakly basic and chemically inert resulted in cleaner reactions with virtually no side products or impurities resulting in a product of superior quality with hardly any complex re-purification steps of the like of recrystallisation or Column chromatography techniques as given in the prior art.
Resins are also non-toxic, stable and possess regenerability properties and also facilitate easy storage, easy handling and easy weighing and are eco-friendly too.

### SUMMARY

The present invention provides a cost-effective and time-efficient, eco-friendly, industrially viable, improved process of preparing aryl fused azapolycyclic compounds of the like of Varenicline of Formula (I)

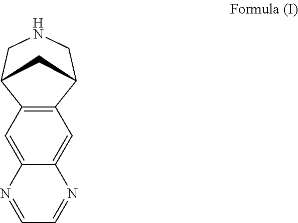

by cyclisation of the corresponding dianiline in the presence of ion exchange resin.

The present invention provides a process for preparing a compound containing a chemical moiety of formula II

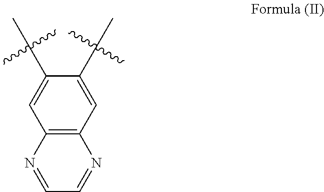

comprising cyclizing a compound containing a chemical moiety of formula III

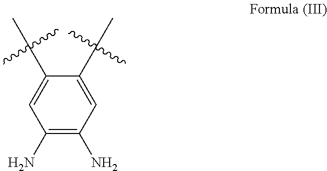

with aqueous glyoxal in a solvent in the presence of an ion exchange resin.

A schematic representation of an improvised process of the present invention is illustrated in Scheme: 1 below:

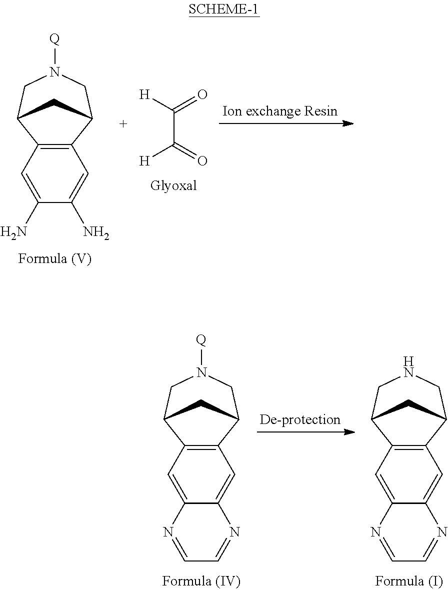

The compound containing the chemical moiety of Dianiline compound of Formula (III) is prepared by known prior art methods or by the process as disclosed in U.S. Pat. No. 6,410,550.

In a preferred embodiment of the present invention is disclosed a process for the synthesis of compound of the formula IV

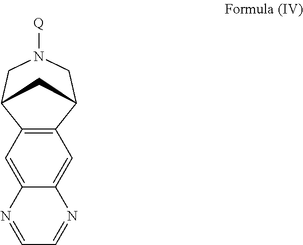

from a compound of Formula (V)

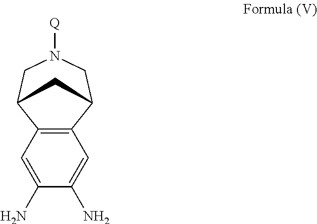

wherein Q is a Nitrogen protecting group selected from the group of trifluoroacetyl group, an acetyl group or a t-butoxy carbonyl group and the like. Preferably, Q is a trifluoroacetyl group, an acetyl group or a t-butoxy carbonyl group. Most preferably, Q is a trifluoroacetyl group.

The compound containing the chemical moiety of N-protected Dianiline compound of Formula (V) is prepared by known prior art methods or by the process as disclosed in U.S. Pat. No. 6,410,550.

In a preferred embodiment of the present invention the compound of Formula (V) is a compound of Formula (VA) whereas the compound of Formula (IV) is a compound of Formula (IVA).

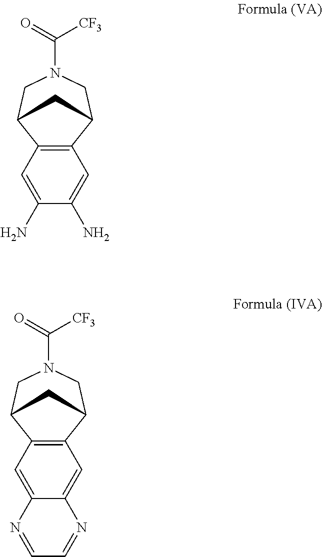

In another embodiment of the present invention is provided a process for preparing a compound of formula (I) or its pharmaceutically acceptable salt thereof comprising the steps of:

(a) Cyclising a compound of Formula (VA) with aqueous Glyoxal in the presence of an ion exchange resin to form the corresponding Quinoxaline of Formula (IVA).

(b) Removing the nitrogen protecting Trifluoroacetyl group by hydrolysis.

(c) Optionally converting the free base from step (b) into its pharmaceutically acceptable salt.

Scheme-2 illustrates an embodiment of the improvised process of the present invention for the preparation of Compound of Formula (I) starting from a compound of Formula (VA).

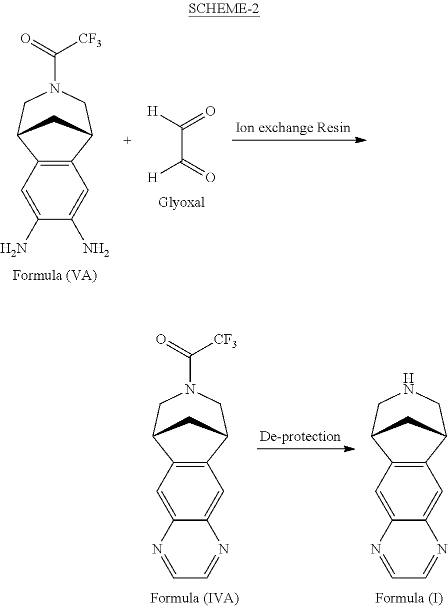

The improvised process of the present invention may be summarized as follows:

A. A process for preparing a compound of formula (I)

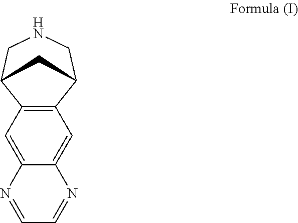

or its pharmaceutically acceptable salt thereof comprising the steps of:

(a)Cyclising a compound of Formula (V),

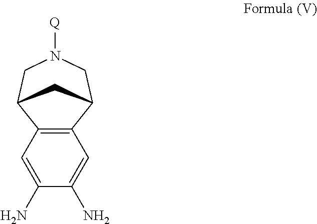

wherein Q is Nitrogen protecting group selected from the group of trifluoroacetyl group, an acetyl group or a t-butoxy carbonyl group and the like,
with aqueous Glyoxal in the presence of an ion exchange resin in a solvent to form the corresponding Quinoxaline of Formula (IV),

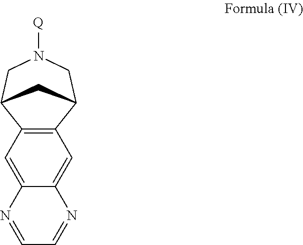

(b)Removing the nitrogen protecting group Q by hydrolysis,

(c) Isolating the compound of formula I as the free base or optionally converting the free base into its pharmaceutically acceptable salt.

B. The process according to step A wherein the ion exchange resin is selected from a group comprising weakly basic, strongly basic, neutral, weakly acidic, strongly acidic types of ion exchange resin and the like or mixtures thereof.

C. The process according to step B wherein the wherein the ion exchange resin may be selected from a group comprising Amberlyst A-21, Amberlite IRA-96 Amberlite IRA-400(Cl), Amberlite IRA-402(OH), Amberlite IRA-410(Cl), Amberlite IRA-900(Cl), Amberlite IRC-748, Ambersep 900(OH). Amberlite IRA-402 Cl, Amberlite IRA-404 Cl, Amberlyst A26 OH, Amberjet 4200 Cl, Amberlite IRC-50, Amberlite IRC-76, Amberlite CG-501 I, Amberlite, IRC-76, Amberlite IRC-86, Amberlite IRA-958 (Cl), Amberlite IRA-910 C, Amberlite IR-120 (H), Amberlite IR-120 (Na), Amberlite-IRA-200(Na), Amberlite IRN-77, Amberlyst-15, Amberlite 200C (Na), Amberlyst 35, Amberlite IRA 67 and the like or mixtures thereof.

D. The process according to step C wherein the ion exchange resin used preferably is Amberlite IRA 67

E. The process according to step A wherein the solvent may be selected from a group comprising alcohols, esters, ethers, amides, nitrites, ketones, hydrocarbon, and the like or non aqueous solvents and the like-or mixtures thereof.

F. The process according to step E wherein the preferred solvent is selected from the group consisting of aqueous alcohol, dioxane, tetrahydrofuran, DMF, DMSO, toluene, and ethyl acetate.

G. The process according to step A wherein the said cyclization may be conducted at a temperature in the range of about −10 ° C. to about boiling point of the solvent.

H. The process according to step A wherein the preferred pharmaceutically acceptable salt is a tartarate salt.

I. The process according to step A wherein the compound of Formula (I) or its pharmaceutically acceptable salt thereof may be prepared by a process comprising the steps of

(a) Cyclising a compound of Formula (VA)

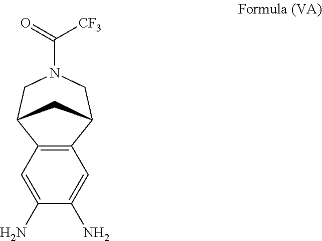

with aqueous Glyoxal in the presence of an ion exchange resin to form the corresponding Quinoxaline of Formula (IVA).

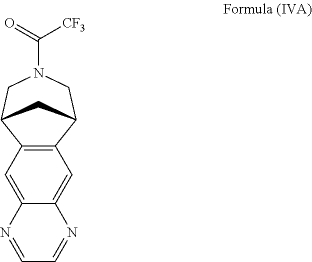

(b) Removing the nitrogen protecting Trifluoroacetyl group by hydrolysis,

(c) Isolating the compound of formula I as the free base or optionally converting the free base into its pharmaceutically acceptable salt.

### BRIEF DESCRIPTION OF DRAWING FIGURES

Further objects of the present invention together with additional features contributing thereto and advantages accruing there from will be apparent from the following description of preferred embodiments of the invention which are shown in the accompanying drawing figures wherein:

Scheme: 1 illustrates a schematic representation of an improvised process of the present invention for the preparation Of Compound of Formula (I) starting from a compound of Formula (V).

Scheme-2 illustrates an embodiment of the improvised process of the present invention for the preparation of Compound of Formula (I) starting from a compound of Formula (VA).

### DETAILED DESCRIPTION OF THE INVENTION

Before the present process and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound”' includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is directed to an improved process for the preparation of substituted quinoxalines by cyclization of the corresponding dianiline in the presence of ion exchange resin. The present invention provides a cost-effective and time-efficient, eco-friendly industrially viable, improved process of preparing aryl fused azapolycyclic compounds of the like of Varenicline of Formula (I)

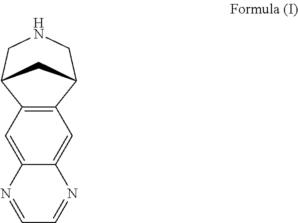

by cyclisation of the corresponding dianiline in the presence of ion exchange resin.

Thus an aspect of the present invention provides a process for preparing a compound containing a chemical moiety of formula II

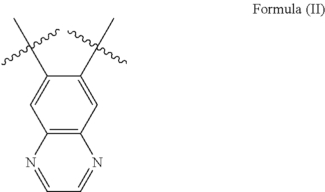

comprising cyclizing a compound containing a chemical moiety of formula III

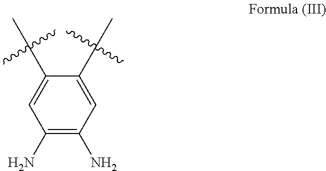

with aqueous glyoxal in a solvent in the presence of an ion exchange resin.

The compound containing the chemical moiety of dianiline compound of Formula (III) is prepared by known prior art methods or by the process as disclosed in U.S. Pat. No. 6,410,550.

In a preferred embodiment of the present invention is disclosed a process for the synthesis of compound of the formula IV

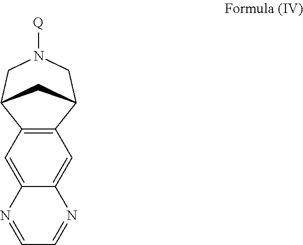

from a compound of Formula (V)

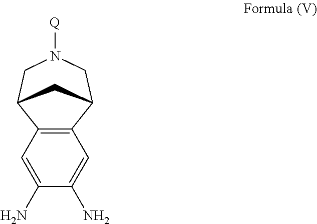

wherein Q is a Nitrogen protecting group selected from the group of trifluoroacetyl group, an acetyl group or a t-butoxy carbonyl group and the like. Preferably, Q is a trifluoroacetyl group, an acetyl group or a t-butoxy carbonyl group. Most preferably, Q is a trifluoroacetyl group.

In a preferred embodiment, the nitrogen protecting group Q is a trifluoroacetyl group.

The compound containing the chemical moiety of N-protected Dianiline compound of Formula (V) may be prepared by known prior art methods or by the process as disclosed in U.S. Pat. No. 6,410,550.

In a preferred embodiment of the present invention the compound of Formula (V) is a compound of Formula (VA) whereas the compound of Formula (IV) is a compound of Formula (IVA)

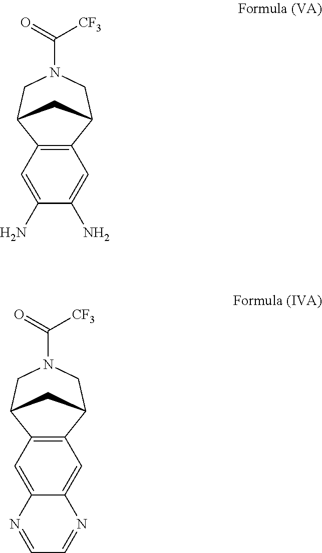

In another embodiment of the present invention is provided a process for preparing a compound of formula (I) or its pharmaceutically acceptable salt thereof comprising the steps of:

(a) Cyclising a compound of Formula (VA) with aqueous Glyoxal in the presence of an ion exchange resin to form the corresponding Quinoxaline of Formula (IVA)

(b) Removing the nitrogen protecting Trifluoroacetyl group by hydrolysis

(c) Optionally converting the free base from step (b) into its pharmaceutically acceptable salt.

According to an aspect of the present invention is provided a process for the preparation of Varenicline of formula I, wherein the ion exchange resin is selected from the group comprising weakly basic, strongly basic, neutral, weakly acidic and strongly acidic resins. The weakly resins may be selected form the group comprising Amberlyst A-21, Amberlite IRA-96 and the like. The strongly basic resins may be selected from the group comprising Amberlite IRA-400(Cl), Amberlite IRA-402(OH), Amberlite. IRA-410(Cl), Amberlite IRA-900(Cl), Amberlite IRC-748, Ambersep 900(OH), Amberlite IRA-402 Cl, Amberlite IRA-404 Cl, Amberlyst A26 OH, Amberjet 4200 Cl and the like. The weakly acidic resins may be selected from the group comprising Amberlite IRC-50, Amberlite IRC-76, Amberlite CG-50 I, Amberlite, IRC-76, Amberlite IRC-86. Amberlite IRA-958 (Cl), Amberlite IRA-910 C and the-like. The strongly acidic resins may be selected from the group comprising Amberlite IR-120 (H), Amberlite IR-120 (Na), Amberlite IRA-200 (Na), Amberlite IRN-77, Amberlyst-15, Amberlite 200C (Na), Amberlyst 35, Amberlite-IRA 67 and the-like:

In a preferred aspect of the present invention, the inventors used resins of the like of Amberlite IRA 67. The cyclisation reaction was thus completed in around 4-5 hrs time duration as against the 18 hours overnight time duration as reported in the prior art processes thus resulting not only in a decreased time duration of the reaction cycle by over 73%, and about 99% product formation with just about 0.5% of unreacted starting material and about 95% practical yield, it also afforded a superior product of superlative purity of around 99.99%.

The cyclisation reaction may be carried out at any temperature between ambient temperature and the boiling point of the solvent. The term ambient temperature in the present application is intended to indicate the temperature preferably in the range of −10° C. to 30° C. In a preferred embodiment of the present invention, the reaction is carried out at a temperature range 0° C. to 30° C.

The cyclisation reaction may be conducted in suitable solvent such as alcohols, esters, ethers, amides, nitrites, ketones, hydrocarbon, and the like or aqueous or non aqueous solvents or mixtures thereof. In a preferred embodiment of the present invention, the preferred solvent for cyclisation is THF.

Removal of the nitrogen protecting group Q may be carried out by methods well known in the art, such as heating with base in a solvent mixture of water and a water immiscible organic solvent including for example, methanol and the like, halogenated hydrocarbons of the like of methylene chloride or aromatic hydrocarbons of the like of toluene, Xylene and the like or mixtures thereof. The base for hydrolysis may be selected from the group comprising sodium or potassium carbonate, sodium or potassium hydroxide and the like or mixtures thereof.

In an embodiment of the present invention, 1-(4,5-Diamino-10-aza-tricyclo[6.3.1.0²⁷]-dodeca-2(7),3,5-triene-10-yl),2,2,2-trifluoroethanone was treated with diluted aqueous 40% Glyoxal solution in the presence of resin in a solvent. After complete conversion was confirmed by HPLC, the reaction solution was filtered and concentrated under vacuum (to a volume of 15-17% of as total volume, diluted with water and the resulting suspension stirred for 1 hr at 10° C., filtered and bed washed with water. The product finally dried at 50° C. at hot air oven to afford 1-(5,8,14-triazatetracycl[10.3.1.0^{2.11}.0^{4.9}]-hexadeca-2(11),3,5.9-pentaene)-2,2,2-trifluoroethanone as a white to pale yellow solid with HPLC Purity: 99.90%

The 1-(5,8,14-triazatetracycl[10.3.1.0^{2.11}.0^{4.9}]-hexadeca-2(11),3,5,9-pentaene)-2,2,2-trifluoroethanone was hydrolysed in Methanol in the presence of base of the like of Potassium carbonate. After the completion of hydrolysis, the base was removed by filtration and the solvent removed under vacuum. The thick residue was dissolved in water and the aqueous phase extracted with MDC. The-organic phase was evaporated under vacuum: the residue was dissolved in methanol and concentrated again. The final residue was dissolved in methanol, treated with activated carbon and filtered through Hyflo to obtain a light brown solution which was preserved for next step.

This solution of the Varenicline free Base in methanol was added to a methanolic solution of tartaric acid at 25-30C in about 1 hour and stirred. The resulting Varenicline tartarate salt was isolated by filtration and dried under vacuum oven to afford Varenicline tartrate as off white to pale-yellow solid.

The term “cyclizing”, as used herein refers to a chemical reaction in which a linear or branched chemical moiety or a substituted ring moiety is converted into a new ring moiety.

The following examples are intended to illustrate the scope of the present invention in all its aspects but not to limit it thereto.

### Examples

Although the invention has been described in terms of particular embodiments and applications: one of ordinary skill in the art, in light of this teaching, can generate additional embodiments and modifications without departing from the spirit of or exceeding the scope of the claimed invention. It should be emphasized that the above-described embodiments of the present invention, particularly any “preferred” embodiments, are merely possible examples of the invention of implementations, merely set forth for a clear understanding of the principles of the invention. Accordingly, it is to be understood that the drawings and descriptions herein are preferred by way of example to facilitate comprehension of the invention and should not be construed to limit the scope thereof.

### Example- 1

### General Procedure for the Preparation of 1-(5, 8, 14-Triazatetracyclo (10.3.1.01-hexadeca-2(11), 3, 5, 9-pentaene)-2, 2, 2-trifluoroethanone

To a 100 ml round bottom flask with thermometer, condenser and stirrer was charged THF (20ml), water (5 ml), resin (1.5 g), stirred and cooled the reaction mass to 0.5° C. Add 1-(4,5-Diamino-10-aza-tricyclo[6.3.0.1]-dodeca-2(7),3,5-trien-10yl)-2,2,2-trifluoroethanone (5 g, 17.5 mmol), under stirring at 0-5° C. A solution of 3.3 g (22.7 mmol) of 40% aqueous Glyoxal diluted with water (11 ml) was added slowly, under stirring at 0-5° C. The resulting solution stirred for 30 min at 0-5° C. and by raising the pot temperature to 25-30° C. stirred for 4-5 hrs. The complete conversion was confirmed by HPLC analysis. The reaction solution was filtered to remove resin for reuse and concentrated under vacuum (30-50 mm of Hg) to a volume of 15-17% of its total volume. Added water (5 ml) and the resulting suspension stirred for 1 hr at 10° C., filter and bed washed with water (25 ml). Suck dry and unload to trays, further dried at 50° C. at hot air oven to obtain the above sample 5.12 g. (95% yield, off white to pale yellow). HPLC Purity: 99.90%

The above experiment was repeated in a similar manner with a number of resins of different types; the results of which are tabulated as under.

### Example-2

### Lab Experimental Data for Various Resin Performance for Cyclization of Diamine Derivative with Glyoxal is Tabulated as Under

| | | | Reaction mass | | | |
|---|---|---|---|---|---|---|
| | | | area Percentage | | | |
| | | | analysis by HPLC | | | |
| | | | Unconverted | | | |
| | | | Starting | Product | % | % |
| | Type | | Material in | formation | Yield | Purity |
| | of | | reaction | in reaction | Ob- | by |
| S.N | Resin | Code No. | mass | mass | tained | HPLC |
| 1 | Weak | Amberlite | ≦0.5% | ≧99.0% | 95% | 99.99% |
| | basic | IRA 67 | | | | |
| 2 | Strong | A200C | 2.7% | 94% | 88% | 93.00% |
| | acidic | | | | | |
| 3 | Weak | IRC-86 | 3.0% | 96% | 82% | 97.00% |
| | acidic | | | | | |
| 4 | Strong | A26OH | 1.5% | 97% | 85% | 98.00% |
| | basic | | | | | |

### Example-3

### Preparation of 5, 8, 14-Triazatetracyclo [10.3.1.0.0]-hexadeca-2(11), 3, 5, 7, 9-pentaene

To a 100 ml round bottom flask with thermometer, condenser and magnetic stirrer was charged Water (10 ml) followed by Potassium carbonate (5 g. 36.17 mmol), stir to get clear solution. Charge methanol(25 ml) and step-I [1-(4,5-Diamino- 10-aza-tricyclo[6.3.1.0]-dodeca-2(7),3,5-trien-10yl)-2,2,2-trifluoroethanone] sample(5 g. 16.20 mmol). Reaction pot heated to 50-55° C. and stirred for 4 hrs. The complete hydrolysis was confirmed by HPLC analysis. The reaction solution concentrated under vacuum (30-50 mm of Hg) at 50° C. The thick residue dissolved in water (15-18 ml ). This aqueous. phase extracted with MDC(26 ml×2). The organic phase was evaporated under vacuum (30-50 mm of Hg) below 40° C. (water bath). the residue was dissolved in methanol (30 ml) and concentrated again (Vacuum: 30-50 mm of Hg, Bath temperature=50° C.). The final residue was dissolved in methanol (50 ml) and 0.1 g of activated carbon was added, the mixture was stirred for 30 min at room temperature. Filtered through Hyflo to obtain light brown solution and it is preserved for next step.

### Example-4

### Preparation of Tartarate Salt of 5, 8, 14-Triazatetracyclo [10.3.1.0.0]-hexadeca-2(11), 3, 5, 7, 9-pentaene

To a 100 ml round bottom flask with thermometer, condenser and magnetic stirrer was charged methanol (30 ml) and L-Tartaric acid (5.07 g, 33.7 mmol), stirred at room temperature for dissolution. Filtered to remove fibrous suspension and filtrate charged back to 100 ml speck-free round bottom flask. Maintaining stirring above methanol solution (step-II) added drop wise at 25-30° C. in 1 hr duration. The reaction solution stirred for 5 hr at 25-30° C., filtered, washed with methanol (10-15 ml) and dried in an oven at 45-50° C. to obtain 5.04 g. Varenicline L-tartarate as a pale yellow solid with HPLC purity: 99.90%

1. A process for preparing a compound of formula (I) 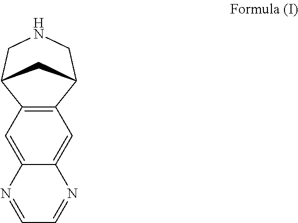
or its pharmaceutically acceptable salt thereof comprising the steps of:
(a) Cyclising a compound of Formula(V) 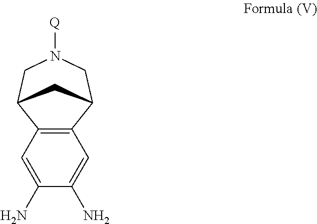
wherein Q is Nitrogen protecting group selected from the group of trifluoroacetyl group, an acetyl group or a t-butoxy carbonyl group and the like;
with aqueous Glyoxal in the presence of an ion exchange resin in a solvent to form the corresponding Quinoxaline of Formula (IV), 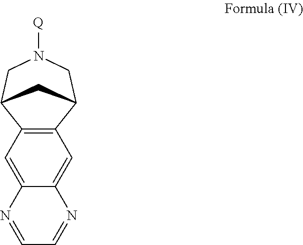
(b) Removing the nitrogen protecting group Q by hydrolysis,
(c) Isolating the compound of formula I as the free base or optionally converting the free base into its pharmaceutically acceptable salt.

2. The process according to claim 1 wherein the ion exchange resin is selected from a group comprising weakly basic, strongly basic, neutral, weakly acidic, strongly acidic types of ion exchange resin and the like or mixtures thereof.

3. The process according to claim 2 wherein the wherein the ion exchange resin may be selected from a group comprising Amberlyst A-21, Amberlite IRA96 Amberlite IRA-400(Cl), Amberlite IRA-402(OH). Amberlite IRA-410(Cl), Amberlite IRA-900(Cl), Amberlite IRC-748, Ambersep 900(OH), Amberlite IRA-402 Cl, Amberlite IRA-404 Cl, Amberlyst A26 OH, Amberjet 4200 Cl, Amberlite IRC-50, Amberlite IRC-76, Amberlite CG-50 I, Amberlite, IRC-76, Amberlite IRC-86, Amberlite IRA-958 (CL), Amberlite IRA-910 C, Amberlite IR-120 (H), Amberlite IR-120 (Na), Amberlite IRA-200 (Na), Amberlite IRN-77, Amberlyst-15, Amberlite 200C (Na), Amberlyst 35, Amberlite IRA 67 and the like or mixtures thereof.

4. The process according to claim 3 wherein the ion exchange resin used preferably is Amberlite IRA 67.

5. The process according to claim 1 wherein the-solvent may be selected from a group comprising alcohols, esters, ethers, amides, nitriles, ketones, hydrocarbon, and the like or non aqueous solvents and the like or mixtures thereof.

6. The process according to claim 5 wherein the preferred solvent is selected from the group consisting of aqueous alcohol, dioxane, tetrahydrofuran, DMF, DMSO, toluene, and ethyl acetate.

7. The process according to claim 1 wherein the said cyclization may be conducted at a temperature in the range of about −10° C. to about boiling point of the solvent.

8. The process according to claim 1 wherein the preferred pharmaceutically acceptable salt is a tartarate salt.

9. The process according to claim 1 wherein the compound of Formula (I) or its pharmaceutically acceptable salt thereof may be prepared by a process comprising the steps of:
(a) Cyclising a compound of Formula (VA) 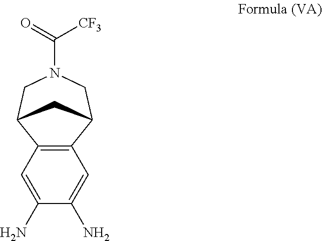
with aqueous Glyoxal in the presence of an ion exchange resin to form the corresponding Quinoxaline of Formula (IVA), 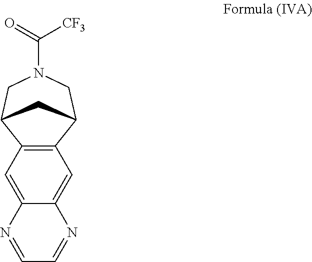
(b)Removing the nitrogen protecting Trifluoroacetyl group by hydrolysis,
(c) Isolating the compound of formula I as the free base or optionally converting the free base into its pharmaceutically acceptable salt.
